(19) United States
(12) Patent Application Publication
(10) Pub. No.: US 2013/0061370 A1
(43) Pub. Date: Mar. 14, 2013

(54) Neck scarf for cooling or warming the user

(71) Applicant: Wiley H. Ezell, Chatham, LA (US)

(72) Inventor: Wiley H. Ezell, Chatham, LA (US)

(21) Appl. No.: 13/573,437

(22) Filed: Sep. 14, 2012

Related U.S. Application Data
(60) Provisional application No. 61/573,899, filed on Sep. 14, 2011.

(51) Int. Cl. A41D 23/00; A61F 7/00

(52) U.S. Cl. 2/207; 607/109

(57) ABSTRACT

A neck scarf which a person can wear to keep himself cool in summer and warm in winter. The scarf's elongated mid-section defines a series of generally rectangular pockets, odd in number, arrayed end-to-end longitudinally for substantially the mid-section's entire length. A single gel pack is slip-fitted into each pocket, which is then sealed shut. Also preparatory to use, each gel pack must be activated: cooling gel packs by placing them in a freezer; warming gel packs by heating them in a microwave. Disposed generally symmetrically about its center pocket, the scarf includes tapered end closure sections used not only to fasten the mid-section snugly about the neck but also to insure the center pocket's gel pack is worn close enough to the hypothalamus to stimulate it, thus magnifying for hours the scarf's effect on its regulation of the body's temperature.

### CROSS REFERENCE TO RELATED APPLICATION

This application is a non-provisional application of the earlier filed provisional application, Ser. No. 61/573,899, filed Sep. 14, 2011, and claims the benefit of the priority of the filing date of Sep. 14, 2011, pursuant to 35 U.S.C. Sec. 119(e).

### FIELD OF THE INVENTION

This invention relates generally to articles of clothing and more specifically to such articles which a person can wear to keep himself cool in hot weather and warm in winter.

### SUMMARY OF THE INVENTION

The object of this invention is to provide, in combination, a neck scarf and a plurality of gel packs which, when activated and mounted within the scarf, transfer heat energy from or, alternately, to the wearer's body, the heat transfer having an effect, which can last for hours, on the body temperature of the neck scarf wearer.

A further object is to provide such a combination in which cooling gel packs, when the scarf is used for cooling, can be activated by placing them in a freezer. Alternately, when the scarf is used for warming, warming gel packs can be activated by heating them in a microwave oven or the like.

The improved scarf, made of two layers of cloth and a heat-reflective, nonwoven liner, defines a series of pockets, each of which is dimensioned in such a way that a gel pack of conventional size can be slip-fitted into the pocket. The scarf's outer cloth layer is preferably fabricated of a waterproof cotton, nylon or polyester fabric. Contiguous with the outer cloth layer is the liner which functions as an insulator. The liner is employed to conserve some of the stored energy of the gel packs. A description of this insulator is found in U.S. Pat. No. 7,452,833. The innermost layer, which in use is positioned next to the wearer's body, is preferably made of the same waterproof fabric as is the outer cloth layer. Closure of each individual pocket, once its gel pack is in place, is achieved with the use of a Velcro® fastener or equivalent such as a zipper, buttons or snaps. Alternately, each of the pockets can be sewn shut after its gel pack has been mounted within it.

Sized to fit around the neck, the scarf's elongated mid-section is the preferred location for the series of pockets in which the gel packs are mounted. Generally rectangular in shape, the pockets are preferably aligned end-to-end, forming an array which extends longitudinally for substantially the entire length of the mid-section.

Further, in the preferred embodiments, the mid-section itself terminates distally in end closure sections which can be fastened together in use, not only holding the mid-section in place about the wearer's neck but also positioning at least one of the gel pack-filled pockets contiguous with the back of the neck. Moreover, when the gel-filled pockets are odd in number (three, preferably), the middle one of them, disposed mid-way between the mid-section's distal ends, tends to be centered on the back of the neck. So centered, the gel pack. in the middle pocket activates the hypothalamus, the gland which regulates an individual's body temperature, and thus magnifies the effect of heat transfer between the improved scarf and the wearer's body. Indeed, one of the reasons why the neck scarf with three gel packs works so well is that the middle gel pack in its array is centered on the back of the neck.

### BRIEF DESCRIPTION OF THE DRAWINGS

FIG. 1 shows a front view of the improved scarf as it is being worn about a user's neck.

FIG. 2 shows the scarf laid flat with its front side up.

FIG. 3 shows the scarf laid flat with its back side up.

FIG. 4 shows the scarf with its end closure sections unfastened from each other.

FIG. 5 shows the scarf in a position in which its end closure sections are fastened together.

FIG. 6 is a closeup view, on an enlarged scale, of a fragmentary portion of the scarf according to FIG. 1, in which one of the scarf's pockets has been opened to receive a gel pack, the gel pack being shown partially inserted into the open pocket.

FIG. 7 is a closeup view of a typical gel pack.

FIG. 8 is a closeup view of a fragmentary portion of the scarf showing the inside surface of its innermost layer as well as the insulator liner.

FIG. 9 is a closeup view, on an enlarged scale, of a fragmentary portion of the scarf with the same open pocket as that shown in FIG. 8.

### DETAILED DESCRIPTION OF THE PREFERRED EMBODIMENT

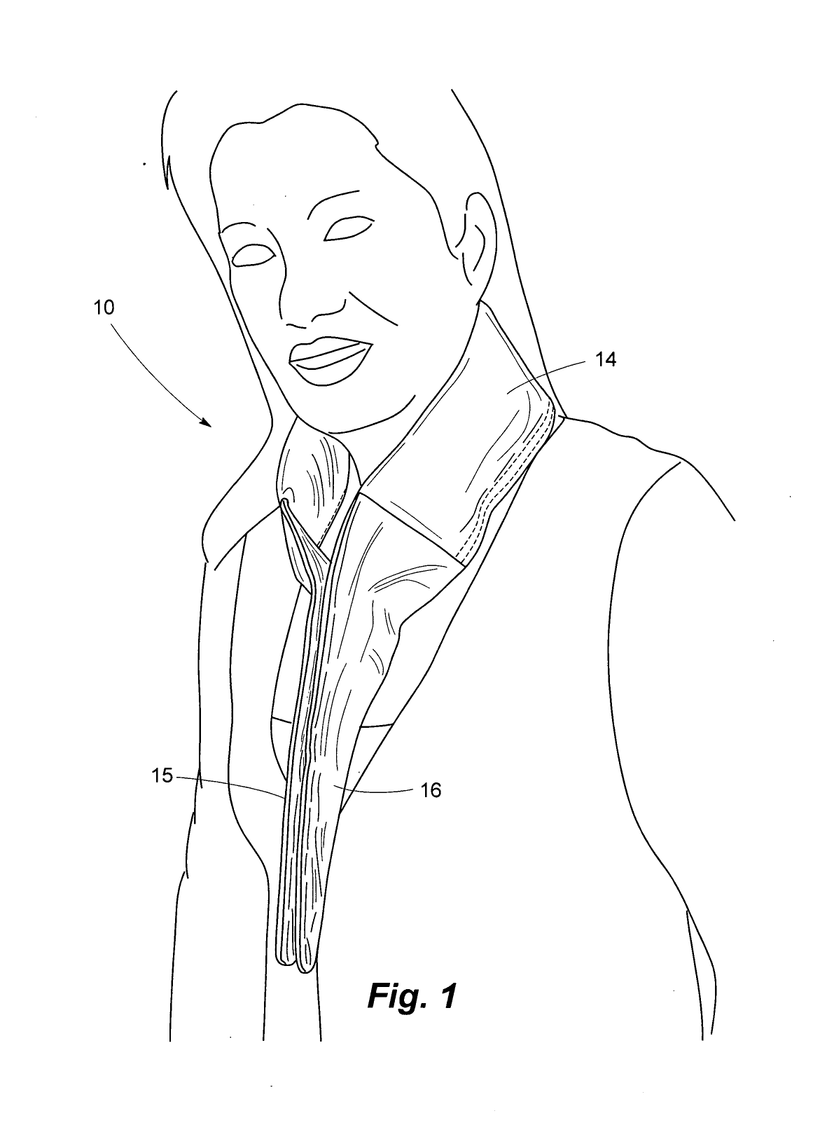

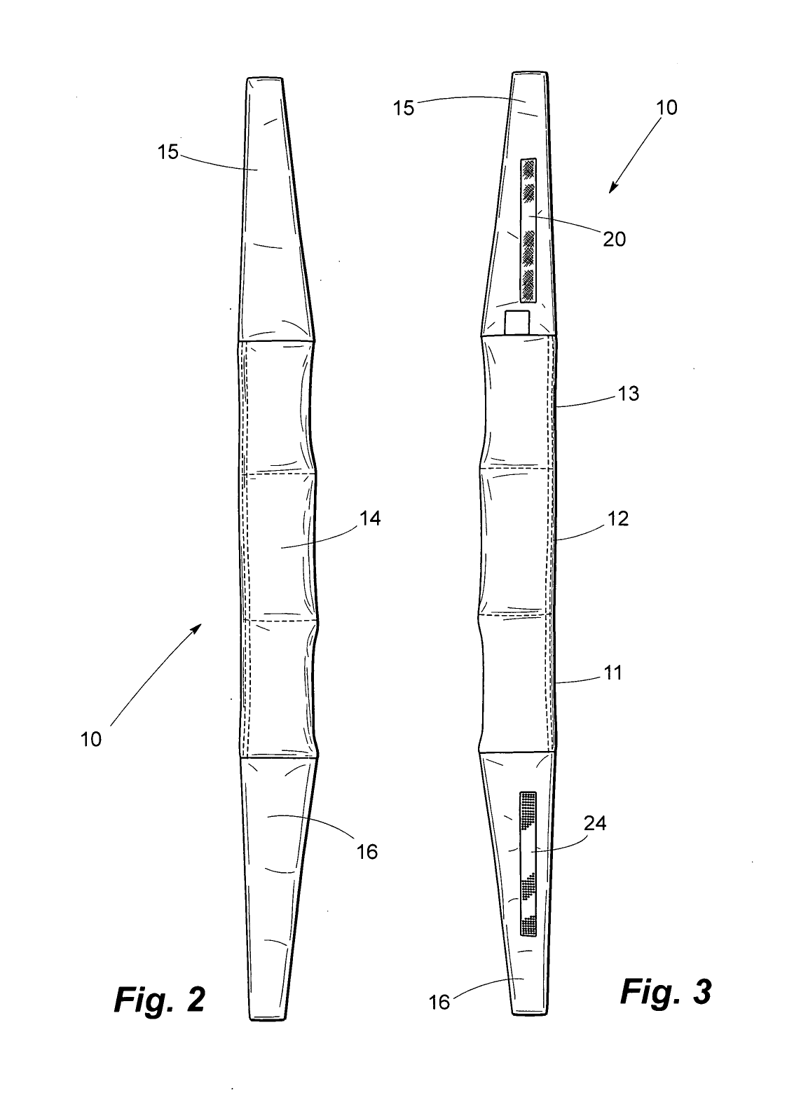

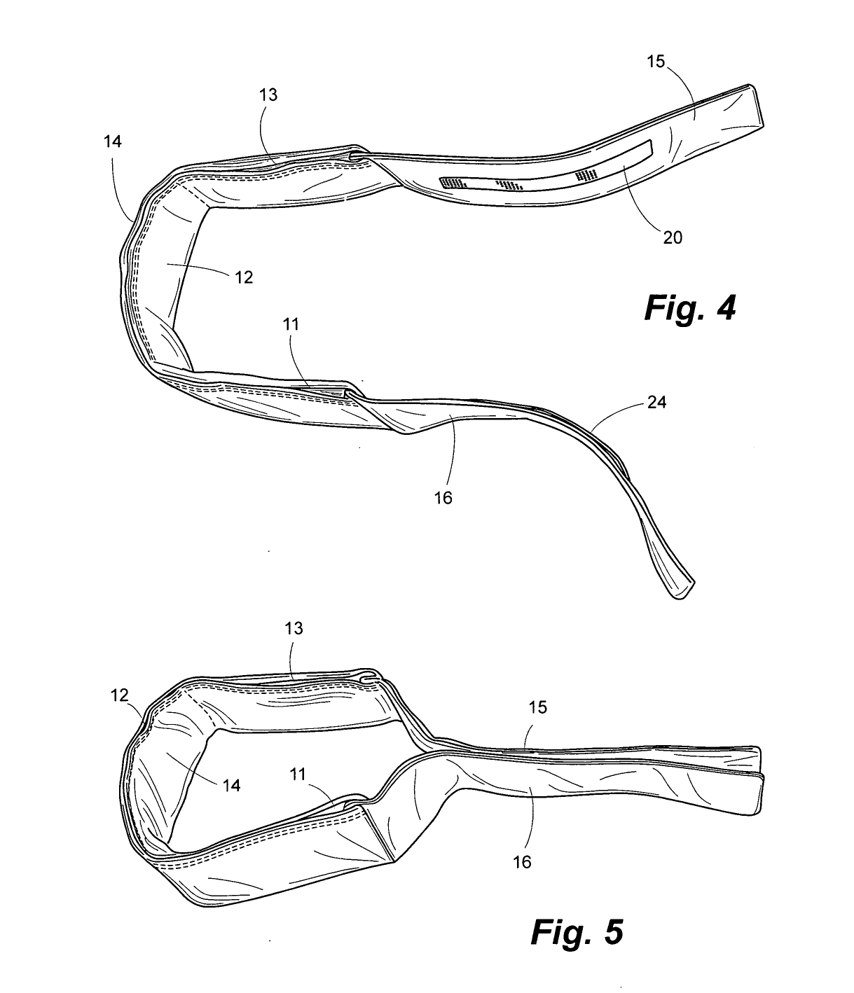

In the drawings, the improved scarf is indicated generally by the reference numeral 10 (FIG. 1). The scarf 10 includes an elongated mid-section 14 which defines an array of generally rectangular pockets 11, 12, 13, which extends longitudinally for substantially the entire length of the mid-section (FIGS. 2 and 3). The mid-section 14 itself terminates distally in tapered end closure sections 15, 16 to which are affixed a mating pair of Velcro® fasteners 20, 24, respectively.

Each pocket 11-13 is sized so that a gel pack 30 such as the Ice-Brik Model IB3, which measures 5 inches long by 2.5 inches wide by 0.5 inch thick, and which is manufactured by Polar Industries, Inc., can be slip-fitted into the pocket. To accommodate an end-to-end array of three pockets 11-13, into each of which a gel pack such as the Ice-Brik Model IB3 can be slip-fitted, the mid-section 14 itself measures about 18 inches in length by 3 inches in width, with each of the pockets 11-13 being about 6 inches long.

For warming, a set of gel packs 30 such as the Model HH2 made by Heatmax Co. of Dalton, Ga. has been found to give good performance for hours when these gel packs are mounted within the pockets 11-13 of the scarf 10 (FIGS. 1-3, 6 and 7). Also, a product known as HotHands made by HeatMax Inc. performs well in the scarf 10.

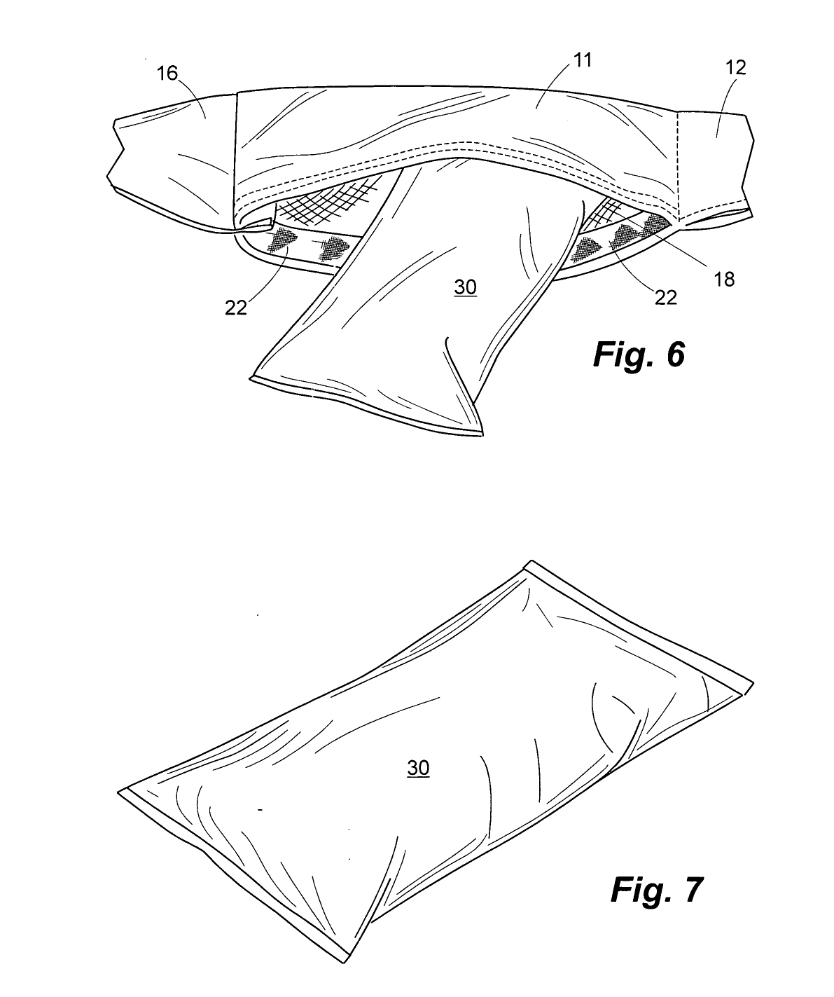

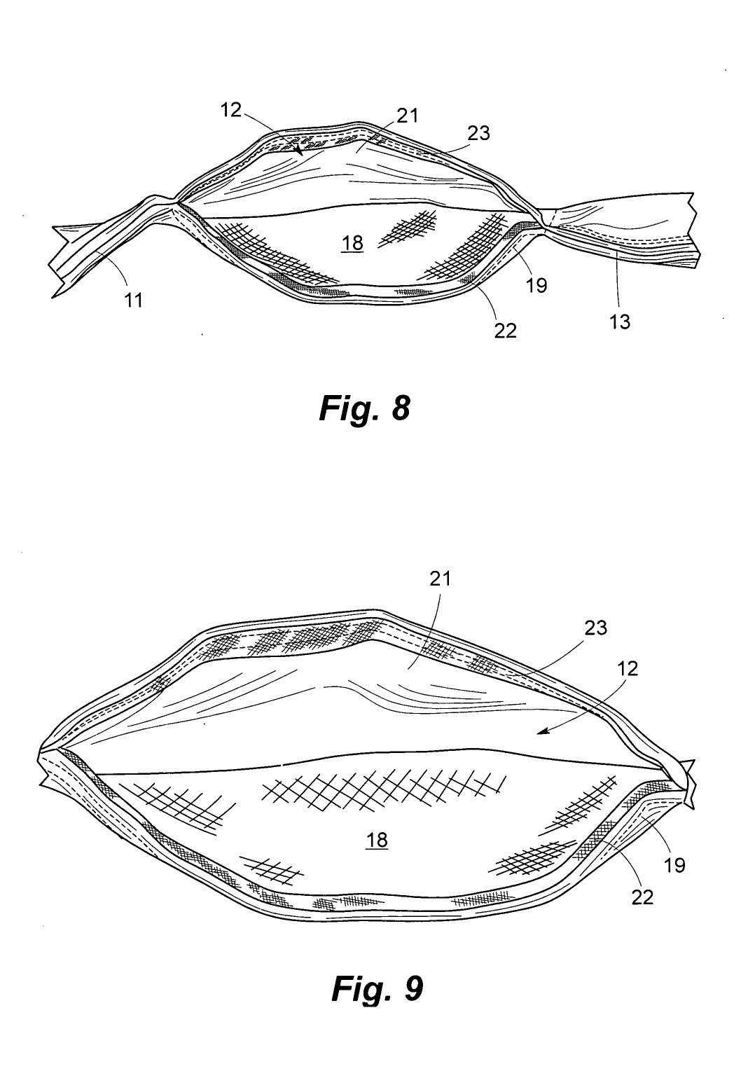

Each of the pockets 11-13 includes two waterproof cloth layers 19, 21 and a layer of insulator 18 (FIGS. 6, 8 and 9). The latter is disposed contiguous with the inside surface of the scarf's layer 19 which, during use, is directed away from the wearer's neck.

As is best illustrated in FIGS. 6, 8 and 9, each pocket includes a pair of mating Velcro® strips 22, 23 which, in the preferred embodiment, are used to seal a removable gel pack 30 within the pocket once the gel pack has been slip-fitted into it. Alternately, to facilitate the use of removable gel packs, zippers, buttons, snaps or the like can be employed to seal the pockets 11-12. Also, the pockets can be sewn shut when removable gel packs are optional.

Generally, the gel packs must be charged just prior to each use. Activation of cooling gel packs, whether or not they are removably housed in the pockets 11-13 of a scarf 10, entails placing them in a freezer. Likewise, activation of warming gel packs such as the Model HH2 requires that they be heated in a microwave oven or the like. Each of the HotHands gel packs, on the other hand, is charged by shaking it vigorously so as to mix the gel pack's contents, causing them to react chemically and release heat.

The end closure sections 15, 16, fastened together by interlocking the Velcro® strips 20, 24 on the back side of these sections, can be used to hold the scarf 10 snugly around the neck (FIGS. 1, 3 and 5). In the preferred embodiment, the end closure sections 15, 16 each measure, by way of example, about 10 inches in length and taper from about 3 inches in width proximate with the scarf's mid-section 14 to about 1 inch in width on each end of the scarf. Together with the mid-section 14, the end closure sections 15, 16 give the scarf 10 an overall length which is preferably about 38 inches.

Importantly, the general symmetry of the scarf 10 with respect to an imaginary line transversely bisecting the center pocket 12 and a gel pack 30 housed within it facilitates a wearer's routinely positioning the latter gel pack in such away that it is both worn in close contact with the back of the neck and close enough to the wearer's hypothalamus that it is stimulated when heat energy is transferred between the center pocket's gel pack and the user's body, thereby magnifying, usually for hours at a time, the scarf's effect on the hypothalamus' regulation of the body's temperature.

1. A neck scarf which a person can wear to keep himself cool in summer, which comprises:
(a) an elongated mid-section which defines a plurality of generally rectangular pockets, the pockets, which are odd in number, being arrayed end-to-end longitudinally for substantially the mid-section's entire length; the pockets so arrayed being disposed, along the mid-section's longitudinal center-line, generally symmetrically about the mid-section's center; the pockets including a middle pocket which is disposed mid-way between the mid-section's distal ends;
(b) a plurality of gel packs, the gel packs containing a gel which can absorb heat for up to hours at a time once the gel has been activated, each gel pack being slip-fitted into, and then individually sealed within, one of the pockets prior to use;
(c) the middle pocket being of sufficient length that it can be held contiguous with the back of the neck and held there in such a way that it spans the spine generally perpendicularly, the middle pocket so held extending past each side of the spine by at least an inch; and
(d) means, including fasteners for holding the mid-section snugly about the neck, for routinely positioning the middle pocket on the back of the neck with said pocket generally centered on the spine, so that transfer of heat away from the wearer's body to the middle pocket's gel pack can stimulate the wearer's hypothalamus for an extended period of time, thus magnifying the scarf's effect on the hypothalamus' regulation of the body's temperature.

2. The neck scarf according to claim 1, wherein each of the pockets includes two layers formed of a waterproof fabric and a heat-reflective liner, the liner being juxtaposed between the two waterproof fabric layers and disposed contiguous with the inside surface of the waterproof fabric layer which is directed away from the wearer's neck in use, the liner functioning as an insulator to conserve some of the heat-absorbent capacity of the gel contained within the gel pack sealed within each individual pocket.

3. The neck scarf according to claim 1, wherein the gel packs are further characterized as being activated by freezing the gel contained within them and in the process releasing the gel's latent heat so that the gel can absorb heat during use.

4. A neck scarf which a person can wear to warm himself in winter, which comprises:
(a) an elongated mid-section which defines a plurality of generally rectangular pockets, the pockets, which are odd in number, being arrayed end-to-end longitudinally for substantially the mid-section's entire length; the pockets so arrayed being disposed, along the mid-sections's longitudinal center-line, generally symmetrically about the mid-section's center; the pockets including a middle pocket which is disposed mid-way between the mid-section's distal ends;
(b) a plurality of gel packs, the gel packs containing a gel which can release heat for up to hours at a time once the gel has been activated, each gel pack being slip-fitted into, and then individually sealed within, one of the pockets prior to use;
(c) the middle pocket being of sufficient length that it can be held contiguous with the back of the neck and held there in such a way that it spans the spine generally perpendicularly, the middle pocket so held extending past each side of the spine by at least an inch; and
(d) means, including fasteners for holding the mid-section snugly about the neck, for routinely positioning the middle pocket on the back of the neck with said pocket generally centered on the spine, so that transfer of heat away from the middle pocket's gel pack to the wearer's body can stimulate his hypothalamus for an extended period of time, thus magnifying the scarf's effect on the hypothalamus' regulation of the body's temperature.

5. The neck scarf according to claim 4, wherein each of the pockets includes a heat-reflective liner and two layers formed of a waterproof fabric, the liner being juxtaposed between the two waterproof fabric layers and disposed contiguous with the inside surface of the waterproof fabric layer which, in use, is directed away from the wearer's neck, the liner functioning as an insulator to conserve some of the heat stored in the gel contained within the gel pack sealed within each individual pocket.

6. The neck scarf according to claim 4, wherein the gel packs are further characterized as being activated by heating the gel contained within them in an oven.

7. A neck scarf which a person can wear to augment the temperature-regulating mechanisms of his own body and, in the process, reduce the amount of climate control-related energy he needs to consume for personal comfort, which comprises:
(a) an elongated mid-section which defines an array of pockets disposed end-to-end longitudinally for substantially the mid-section's entire length, the pockets including a middle pocket which is disposed mid-way between the mid-section's distal ends;
(b) a plurality of gel packs, the gel packs containing a gel which, once activated, can transfer heat between the scarf wearer's body and the gel for up to hours at a time, each gel pack being individually sealed within one of the pockets prior to use; and
(c) means, including longitudinal extensions of the mid-section and a pair of spaced apart releasable fasteners, each fastener being affixed to one of the extensions and disposed generally equidistant from the middle pocket, for holding the mid-section snugly about the neck while the middle pocket is approximately centered with respect to the spine, the middle pocket being sufficiently long that when it is so held, it extends past each side of the spine by at least one inch, so that transfer of heat between the wearer's body and the middle pocket's gel pack can stimulate the wearer's hypothalamus for an extended period of time, thus magnifying the scarf's effect on the hypothalamus' regulation of the body's temperature.
